(19) United States
(12) Patent Application Publication
(10) Pub. No.: US 2014/0324073 A1
(43) Pub. Date: Oct. 30, 2014

(54) SURGICAL CLAMP APPARATUS AND A SURGICAL CLAMP FOR USE IN KEYHOLE SURGERY

(71) Applicant: Cook Medical Technologies LLC, Bloomington, IN (US)

(72) Inventors: Michael C. Junger, Queensland (AU); Tiffany Burleigh, Queensland (AU); Andrew Robert Leslie Stevenson, Queensland (AU)

(73) Assignee: Cook Medical Technologies LLC, Bloomington, IN (US)

(21) Appl. No.: 14/189,390

(22) Filed: Feb. 25, 2014

(30) Foreign Application Priority Data
Apr. 30, 2013 (AU) 2013205730

(51) Int. Cl. A61B 17/122; A61B 17/128

(52) U.S. Cl. CPC A61B 17/122; A61B 17/128
USPC 606/142; 606/157

(57) ABSTRACT

A surgical clamp for forming a clamping loop around a bodily lumen is disclosed. The clamp is deliverable through keyhole surgery. The clamp comprises: an elongate body comprising an elongate deformable layer having a first hardness and an elongate core having a second hardness, the first hardness less than the second hardness; a leading portion comprising a gripper, the gripper having an open mouth gripably engageable with the deformable layer to form the clamping loop; and a trailing portion. The elongate body joins the trailing portion to the leading portion. The elongate body is biased to move from a constrained generally straight condition within a delivery device to an unrestrained curled condition within a patient.

### RELATED APPLICATIONS

The present application claims the benefit of priority to Australian Patent Application No. 2013205730, filed Apr. 30, 2013, and entitled “A Surgical Clamp Apparatus And A Surgical Clamp For Use In Keyhole Surgery,” the entire contents of which is incorporated herein by reference.

### FIELD OF THE INVENTION

The invention relates generally to medical devices and, in particular to surgical clamps that are deployable through keyhole surgery.

### BACKGROUND

Temporary ligation of a tubular lumen is often required during surgical procedures. For instance, ligation of a bowel lumen, a blood vessel or other bodily lumens, vessels or tubes during surgery may be required. Temporary ligation of lumens is achieved by a number of methods currently. For instance, in gastrointestinal surgery for the resection of colorectal cancer and for the manipulation and retraction of bowel lumen during laparoscopic lower pelvic dissection, surgeons currently use nylon tape or sutures to isolate the lumen. The tape or suture method requires considerable skill and is time consuming.

A further problem with existing ligation tools and methods is that often an additional assistant is required. For instance an additional assistant may be required during gastrointestinal surgery on females to manipulate the uterus interiorly so as to improve the access and visual operative fields in which the surgeon has to operate in achieving ligation.

It is an object of the present invention to provide a surgical clamp that can be deployed in keyhole surgery that ameliorates at least some of the aforementioned problems or at least offer a useful choice.

### SUMMARY

A surgical clamp for forming a clamping loop around a bodily lumen, the clamp being deliverable through keyhole surgery is described. In one example, the clamp comprises an elongate body comprising an elongate deformable layer having a first hardness and an elongate core having a second hardness, the first hardness less being than the second hardness. The clamp further comprises a leading portion comprising a gripper, the gripper having an open mouth gripably engageable with the deformable layer to form the clamping loop, a trailing portion wherein the elongate body joins the trailing portion to the leading portion, and wherein the elongate body is biased to move from a constrained generally straight condition within a delivery device to an unrestrained curled condition within a patient.

In one example the open mouth of the gripper is shaped to allow lateral entry of the elongate body into the gripper.

In one example the open mouth of the gripper is arranged and constructed so as to provide a higher degree of slip resistance against movement of the deformable layer in a direction loosening the clamping loop than the degree of slip resistance against movement of the deformable layer in a direction tightening the clamping loop.

In one example the open mouth comprises a tooth and a face opposing the tooth together defining an opening, wherein the opening is sized to grippingly receive the deformable layer.

In one example the tooth is asymmetrically shaped so as to provide the higher degree of slip resistance against movement of the deformable layer in a direction loosening the clamping loop than the degree of slip resistance against movement of the deformable layer in a direction tightening the clamping loop.

In one example the tooth of the mouth is wedge shaped.

In one example the deformable layer has an external shape that defines a series of ratchet teeth, in use the ratchet teeth co-operable with the tooth of the mouth to form a ratchet.

In one example the elongate core is formed from a super elastic material.

A surgical clamp assembly for use in keyhole surgery is also described. In one example, the assembly comprises a surgical clamp for forming a clamping loop around a bodily lumen, wherein the clamp comprises an elongate body. The assembly further comprises a deployment tube for deploying the clamp through a keyhole and a manipulator for manipulating the clamp through the deployment tube, wherein the elongate body is biased to move from a constrained generally straight condition within the deployment tube to an unrestrained curled condition within a patient.

In one example the clamp comprises a leading portion comprising a gripper, the gripper having an open mouth gripably engageable with the elongate body to form the clamping loop and a trailing portion, wherein the elongate body joins the trailing portion to the leading portion, and wherein the elongate body is biased to move from a constrained generally straight condition within a delivery device to an unrestrained curled condition within a patient.

In one example the open mouth of the gripper is shaped to allow lateral entry of the elongate body into the gripper.

In one example the open mouth of the gripper is arranged and constructed so as to provide a higher degree of slip resistance against movement of the elongate body in a direction loosening the clamping loop than the degree of slip resistance against movement of the elongate body in a direction tightening the clamping loop.

In one example the open mouth includes a tooth and a face opposing the tooth together defining an opening, wherein the opening is sized to grippingly receive the elongate body.

In one example the assembly further comprises a latching arrangement to latch the clamp to the manipulator.

In one example the latching arrangement comprises a first latch surface on the manipulator co-operable with a second latch surface on the clamp,

wherein the first and second latching surfaces remain engaged while the second latch surface remains inside the deployment tube and wherein the first and second latching surfaces readily disengage when the second latch surface is outside the deployment tube.

In one example the first latch surface is located on a latching projection that projects from the manipulator and the second latch surface is located within a latching slot within the clamp.

In one example the latching arrangement comprises a locking arrangement, the locking arrangement preventing substantial axial movement of the manipulator in a direction axial to the deployment tube and hence preventing unintended release of the clamp.

In one example the tooth is asymmetrically shaped so as to provide the higher degree of slip resistance against movement of the elongate body in a direction loosening the clamping loop than the degree of slip resistance against movement of the elongate body in a direction tightening the clamping loop.

In one example the tooth of the mouth is wedge shaped.

In one example the elongate body has an external shape that defines a series of ratchet teeth, in use the ratchet teeth co-operable with the tooth of the mouth to form a ratchet.

In one example the elongate body comprises an elongate deformable layer having a first hardness and an elongate core having a second hardness, the first hardness less that the second hardness;

In one example the elongate core is formed from a super elastic material.

### DESCRIPTION OF THE DRAWINGS

FIGS. 1a, 1b, 1c and 1d are diagrammatic perspective views showing a surgical clamp assembly and surgical clamp according to an embodiment of the invention.

FIG. 2a is an isometric view of the surgical clamp assembly FIG. 1a.

FIG. 2b is a side view of the assembly of FIG. 2a.

FIG. 2c is a close up view of a portion of the clamp assembly shown in FIG. 2a.

FIG. 3a is an isometric view of a manipulator that forms part of the clamp assembly of FIG. 2a.

FIG. 3b is a side view of the manipulator of FIG. 3a.

FIG. 4a is an isometric view of a surgical clamp according to the invention.

FIG. 4b is a side view of the surgical clamp of FIG. 4a.

FIG. 4c is a cross-sectional view through section lines 4c-4c as indicated on FIG. 4b.

FIG. 4d is a close up view of a portion of the surgical clamp shown in FIG. 4a.

FIG. 4e is a cross-sectional view through section lines 4e-4e as indicated on FIG. 4b.

FIGS. 5a, 5b and 5c show an alternative surgical clamp according to the invention in similar views to that of FIGS. 4b, 4d and 4e.

FIG. 6 is a diagrammatic perspective view showing deployment of the surgical clamp of the invention around a bowel lumen within the abdominal bowel cavity.

### DETAILED DESCRIPTION

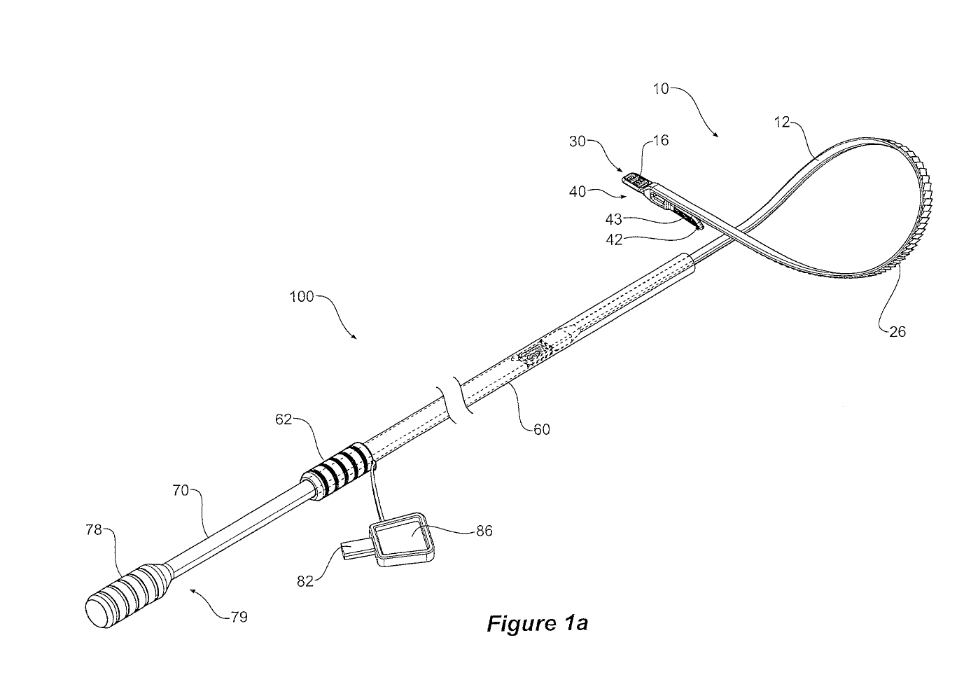

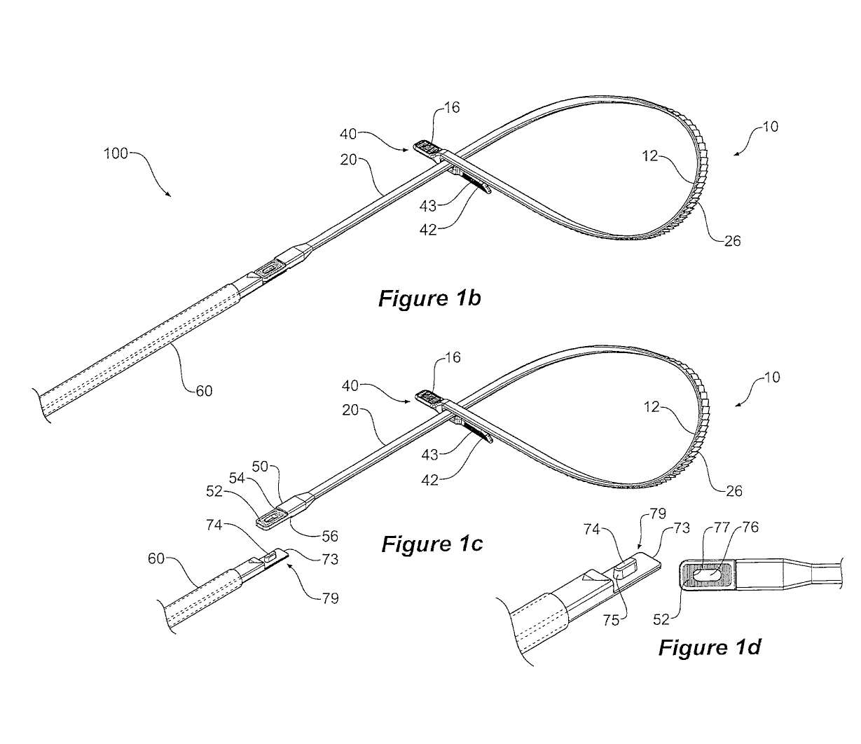

FIGS. 1a, 1b and 1c show a surgical clamp assembly for use in keyhole surgical. The assembly allows deployment of a surgical clamp through a conventional laparoscopic port.

The surgical clamp assembly 100 includes a surgical clamp 10 for forming a clamping loop around a bodily lumen (such as a bowel lumen), a deployment tube 60 for deploying the clamp 10 through a keyhole and a manipulator 70 for manipulating the clamp through the deployment tube.

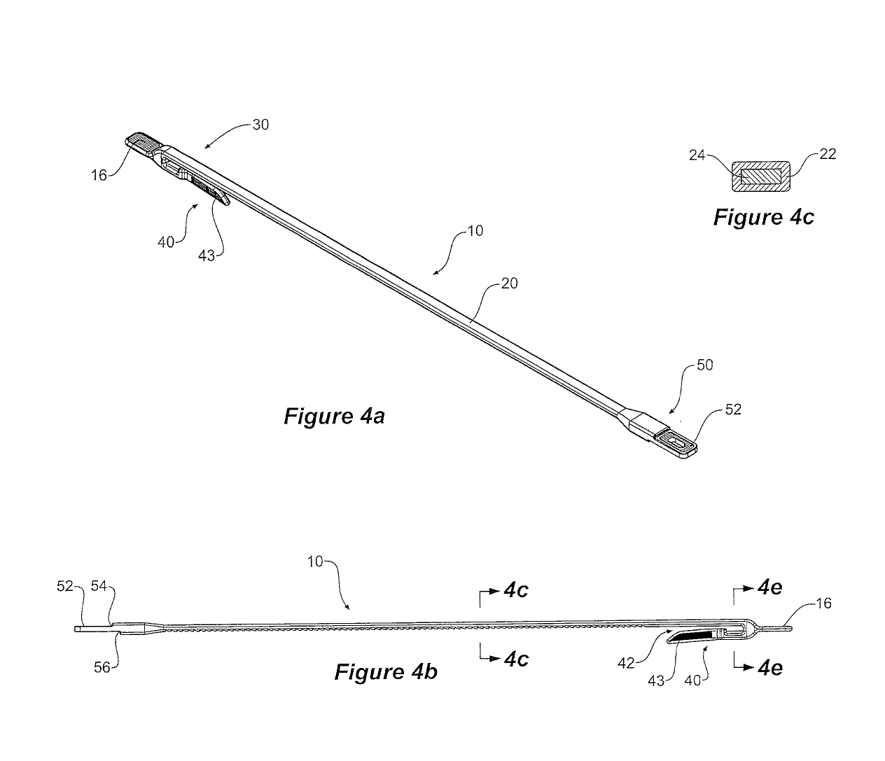

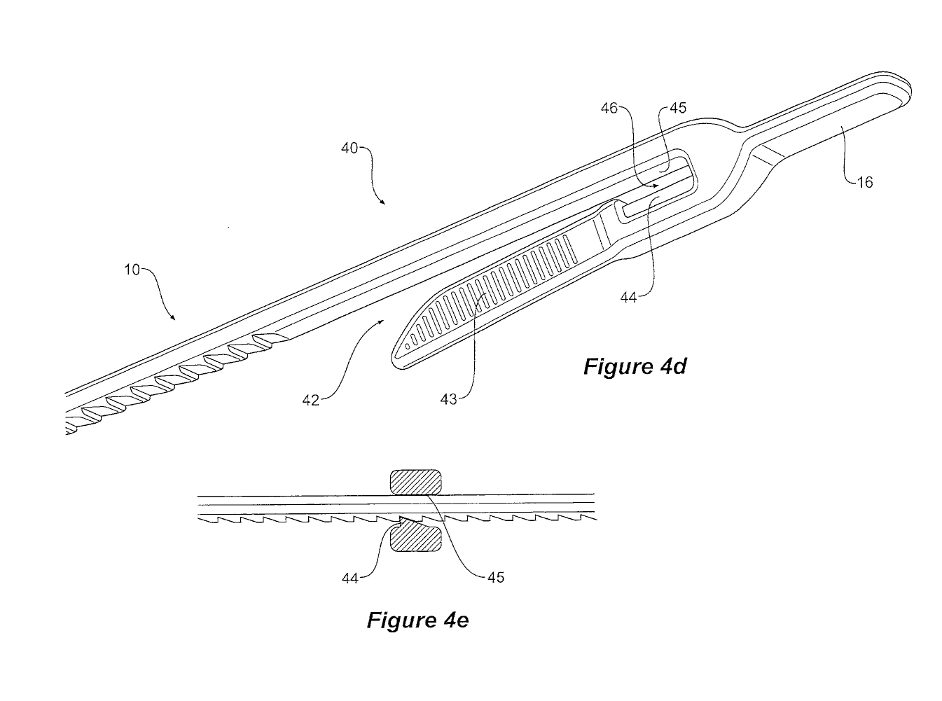

The surgical clamp 10 is shown more clearly in FIGS. 4a, 4b and 4c. Referring to these figures, it can be seen that the surgical clamp 10 includes an elongate body 20, a leading portion 30 that includes a gripper 40 and a trailing portion 50. The elongate body 20 joins the trailing portion 50 to the leading portion 30. The elongate body 20 includes an elongate deformable layer 22 and a elongate core 24 shown most clearly in the cross-sectional view of FIG. 4c taken through section lines 4c-4c shown in FIG. 4b.

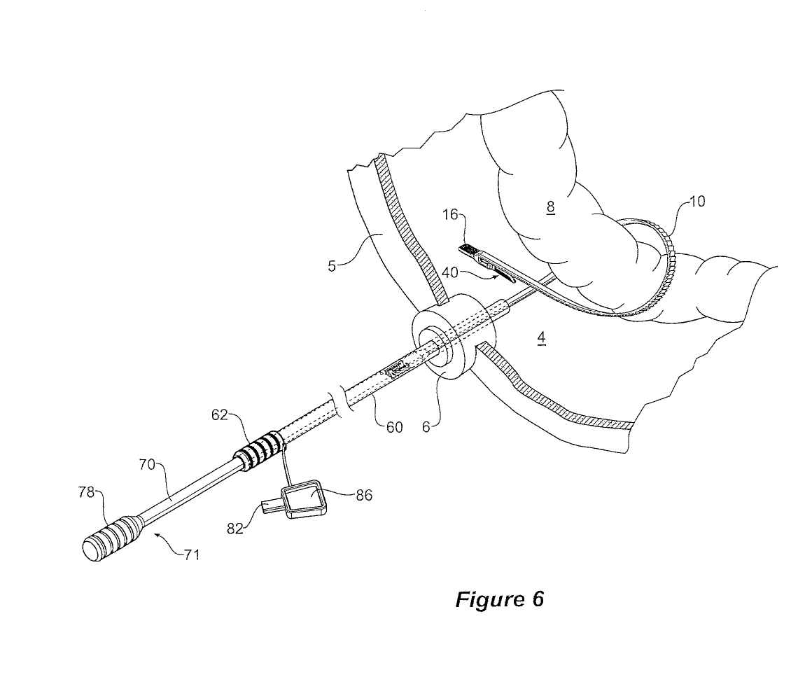

The gripper 40 of the leading portion 30 has an open mouth 42 as shown in FIGS. 4b and 4d. The open mouth 42 is shaped to allow lateral entry of the elongate body 20 into the gripper. The open mouth 42 is gripably engageable with the deformable layer 22 to form a clamping loop as is illustrated in FIGS. 4e and 6.

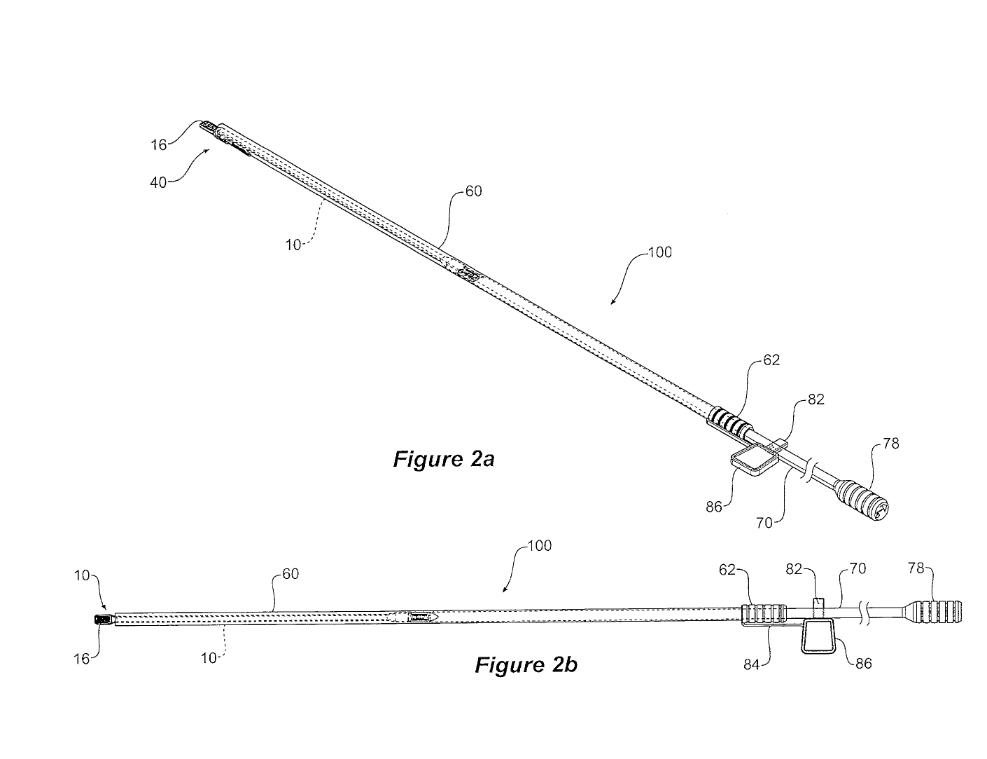

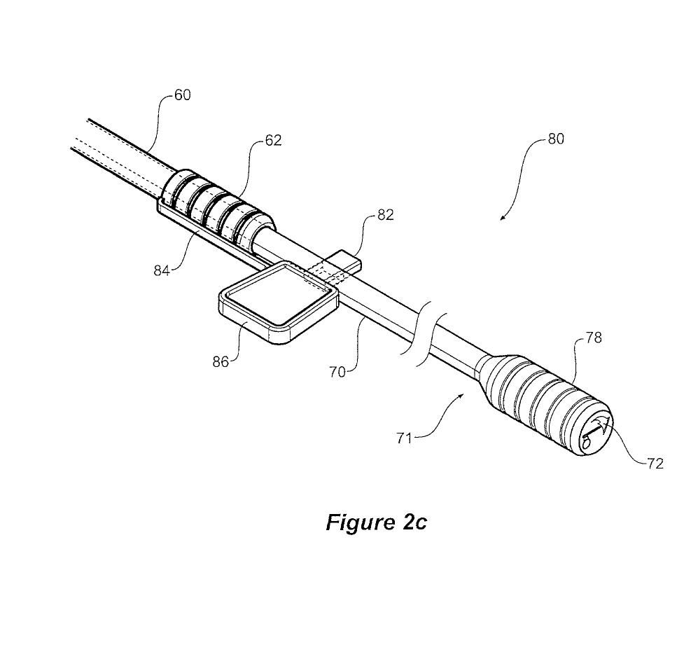

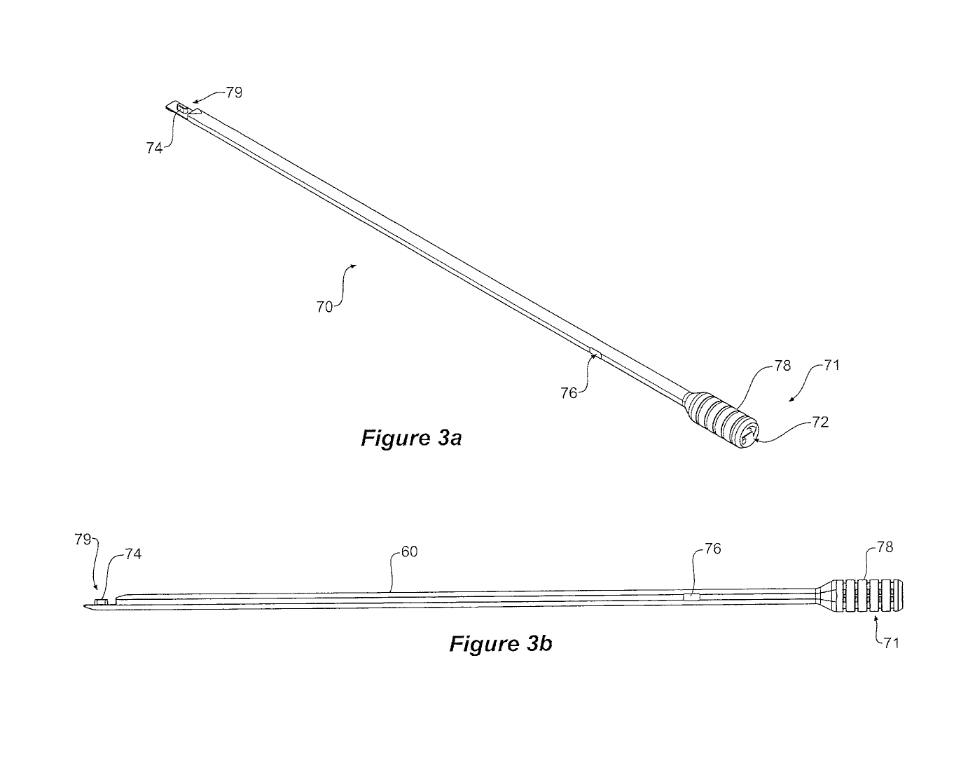

FIG. 4a shows the surgical clamp 10 in an extended approximately linear condition for clarity. While the clamp 10 sits in this condition within the deployment tube 60 as shown in FIGS. 2a and 2b, it is biased to move from this generally straight condition, constrained within the tube 60, to an unrestrained curled condition within a patient such as is shown in FIG. 6. More specifically, the surgical clamp 10 is biased such that it will curl around its target lumen, such as a bowel or blood vessel, as it is deployed, as is shown in FIG. 6. When fully deployed, the gripper 40 is substantially aligned with the elongate body 20 as can be seen in FIGS. 1 b and 1c.

The gripper 40 on the clamp 10 will now be described in more detail with reference to FIGS. 4b, 4d and 4e. The open mouth 42 of the gripper 40 is arranged and constructed so as to provide a higher degree of slip resistance against movement of the deformable layer in a direction loosening the clamping loop 12 than the degree of slip resistance against movement of the deformable layer in a direction tightening the clamping loop 12. More specifically, the open mouth 42 includes a tooth 44 and a face 45 opposing the tooth 44 as is most clearly shown in FIG. 4e. The tooth 44 and face 45 together define an opening 46 as shown in FIG. 4d, wherein the opening 46 is sized to grippingly receive the deformable layer 22. A mouth tab 43 also forms part of the gripper 40. The mouth tab 43 is grippable by a standard surgical gripper and allows the surgeon to release the gripper so as to loosen or remove the clamp 10.

The tooth 44 is asymmetrically shaped so as to provide the higher degree of slip resistance against movement of the deformable layer in the direction of loosening the clamping loop 12 than the degree of slip resistance against movement of the deformable layer in a direction tightening the clamping loop 12. It can be seen from the cross-sectional view of FIG. 4e that the tooth is wedged shaped.

The elongate core 24 of the clamp 10 can be formed from a super elastic material. For instance, Nitinol. The deformable layer 22 of the clamp 10 can be formed from silicon. The silicon may be over moulded over the Nitinol strip. The trailing portion 50 and the leading portion 30 of the clamp 10 may have the same Nitinol core but may be over moulded with alternative plastics such as polycarbonate, polypropylene or similar materials. The leading portion 30 includes a leading tab 16 suitable for gripping with a surgical gripper. Similarly, a trailing tab 52 is provided at the trailing portion 50 of the clamp 10. Again, the trailing tab is suitable for gripping with a surgical gripper. The leading and trailing tabs allow a surgeon to readily manoeuvre the clamp 10 during a procedure.

The surgical clamp assembly 100 includes a latching arrangement to latch the clamp 10 to the manipulator 70. The latching arrangement is shown in FIGS. 1a, 1b, 1c and 1 d. FIG. 1c shows the distal end 79 of the manipulator 70 extended out through the end of the deployment tube 60. It can be seen from FIGS. 1c and 1d that the distal end 79 of the manipulator has a latching projection 74 that has a latching surface 75 for engagement with a corresponding latching surface 77 inside a latching slot 76 within the clamp 10 as is most clearly shown at FIG. 1 d.

Referring to FIGS. 1c and 1d, it can be seen that the trailing portion 50 of the clamp has shoulders 54 and 56 on opposing sides. The shoulders are sized such that the trailing portion 50 can only fit onto the projection 74 at the end of the manipulator 70 one way. If an attempt is made to assemble the surgical clamp assembly 100 with the clamp 10 upside down as compared to that shown in FIG. 1 c, the end 73 of the manipulator 70 will not align with the shoulder 54. This prevents incorrect assembly.

Referring to FIG. 1a and FIGS. 2a and 2b, it can be seen that the latching surfaces 75 and 77 remain engaged while the latching surface 77 remains inside the deployment tube 60. Once the manipulator has been extended out from the deployment tube 60 as is shown in FIG. 1 b, the clamp 10 can release from the manipulator 70.

Referring again to FIGS. 2a, 2b and 2c, it can be seen that the latching arrangement of the surgical clamp assembly 100 has a locking arrangement 80 to prevent premature deployment of the clamp 10. The locking arrangement prevents substantial axial movement of the manipulator 70 in a direction axial to the deployment tube 60 and therefore prevents unintended release of the clamp 10 from the manipulator 70. The locking arrangement 80 comprises a pin 82 engaged in a slot 76 within the manipulator 70 and a pin retainer 84. The pin 82 prevents substantial movement of the manipulator 70 in a direction axial to the deployment tube 60. The locking arrangement 80 is shown more clearly in FIG. 2c. It can be seen that the pin 82 has a grippable handle 86. The tube 60 has an end piece 62 and a pin retainer 84 which extends between the tube end piece 62 and the pin handle 86. FIG. 2c also shows an arrow symbol on the proximal end 71 of the manipulator 70. The arrow symbol 72 is provided to indicate to the surgeon the deployment direction for the clamp 10.

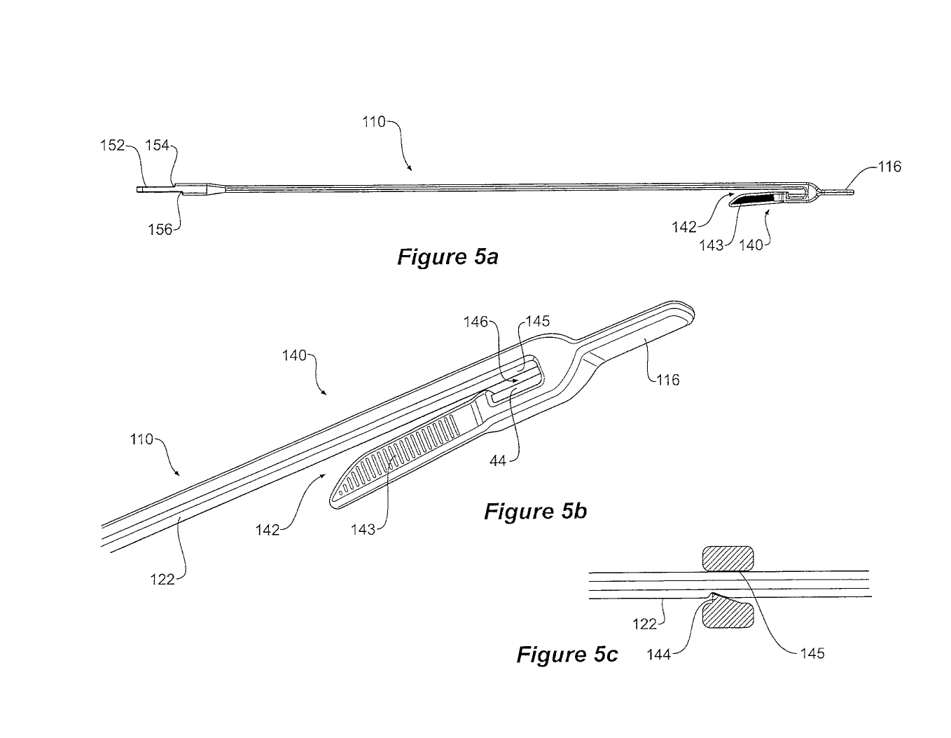

As can be seen in FIG. 4b, the deformable layer 22 of the clamp 10 has an external shape that defines a series of ratchet teeth. The ratchet teeth co-operates with the tooth 42 to form a ratchet. FIGS. 5a, 5b and 5c show an alternative embodiment of the invention. The clamp 110 is similar to the clamp 10 shown in FIG. 4b but the deformable layer 122 is smooth and flat. It is locally deformed by the tooth 144 within the gripper 140. Other embodiments, not shown may have deformable layers with a combination of teeth serrations and smooth sections for instance.

Operation of the surgical clamp assembly 100 will now be described with reference to FIGS. 1a, 1b, 1c, 1d, 2a, 2b and 6. Referring first to FIGS. 2a and 2b, the surgical clamp assembly 100 is shown prior to use. In this configuration, the surgical clamp 10 is housed primarily within the deployment tube 60. It is latched to the manipulator 70. A pin 82 that passes through a slot within the manipulator 70 holds the manipulator to the deployment tube 60. This in turn prevents axial movement of the clamp 10 with respect to the deployment tube 60.

A surgeon can take the assembly 100 as illustrated in FIGS. 2a and 2b and insert it through a conventional laparoscopic port 6 such as is diagrammatically illustrated in FIG. 6. This enables the surgeon to access a bodily cavity 4 from a position external to the body of the patient.

When the surgeon is ready to commence deployment of the clamp 10, he/she removes the pin 82 using the pin handle 86 from the slot 76 within the manipulator 70. He/she is then able to manipulate the handle 78 as the manipulator 70 in both an axial and rotational direction. Through manipulation of the handle 78, the surgeon can control the deployment of the clamp 10 such that it curls around a bodily lumen, such as a bowel 8 that is shown in FIG. 6. As described previously, the clamp 10 is biased to curl into the position shown in FIG. 6. If necessary, the surgeon may grip the leading tab 16 at the leading end of the clamp 10 in order to assist in circling the bowel 8. The surgeon can also grip the mouth tab 43 most clearly illustrated in FIG. 4d to assist with engaging the gripper 40 with the deformable layer 22 as is shown in FIG. 1 b. The open mouth 42 of the gripper 40 facilitates easy lateral entry of the elongate body 20 of the clamp 10 into the gripper 40. The gripper 40 is also shaped such that the surgeon can easily loosen or release its grip on the elongate body 20.

The surgeon may use the indicator arrow 72 best shown in FIG. 2c to show the orientation of the clamp 100.

When the surgeon wishes to release the clamp 10 from the deployment tube 60, he/she slides the handle 78 of the manipulator 70 all the way in until it contacts the end tubing piece 62. FIG. 1 b shows this position at the proximal end of the deployment tube 60. As is shown progressively in FIGS. 1b and 1 c, in this position, the clamp 10 is able to release from the manipulator 70.

While the clamp 10 may be used as part of the surgical clamp assembly 100 as described above, alternatively it may be used on its own. For instance the clamp 10 may be used in open surgery without the delivery system.

The embodiments of the invention described above reduces the skill required to feed the clamp around the vessel and/or to tie it off. It also provides a less traumatic closure than a suture.

Throughout this specification, the term distal with respect to the deployment device or prosthesis means the end the deployment device or the prosthesis further away in the direction from the patient, while the deployment device or the prosthesis is in use, and the term proximal means the portion of the deployment device or the prosthesis nearer to the patient, while the deployment device or the prosthesis is in use.

It is to be understood that the above described embodiments of the invention are merely illustrative of the principals of this invention and that other surgical clamping assemblies and surgical clamps may be devised by those skilled in the art without departing from the spirit and scope of this invention.

1. A surgical clamp for forming a clamping loop around a bodily lumen, the clamp deliverable through keyhole surgery, the clamp comprising:
an elongate body comprising an elongate deformable layer having a first hardness and an elongate core having a second hardness, the first hardness less than the second hardness;
a leading portion comprising a gripper, the gripper having an open mouth gripably engageable with the deformable layer to form the clamping loop; and
a trailing portion, the elongate body joining the trailing portion to the leading portion,
wherein the elongate body is biased to move from a constrained generally straight condition within a delivery device to an unrestrained curled condition within a patient.

2. The clamp of claim 1 wherein the open mouth of the gripper is configured to allow lateral entry of the elongate body into the gripper.

3. The clamp of claim 2 wherein the open mouth of the gripper is configured to provide a higher degree of slip resistance against movement of the deformable layer in a direction loosening the clamping loop than the degree of slip resistance against movement of the deformable layer in a direction tightening the clamping loop.

4. The clamp of claim 3 wherein the open mouth includes a tooth and a face opposing the tooth together defining an opening, wherein the opening is sized to grippingly receive the deformable layer.

5. The clamp of claim 4 wherein the tooth is asymmetrically shaped so as to provide the higher degree of slip resistance against movement of the deformable layer in a direction loosening the clamping loop than the degree of slip resistance against movement of the deformable layer in a direction tightening the clamping loop.

6. The clamp of claim 5 wherein the tooth of the mouth is wedge shaped.

7. The clamp of claim 6 wherein the deformable layer has an external shape that defines a series of ratchet teeth and wherein the ratchet teeth are cooperable with the tooth of the mouth to form a ratchet.

8. The clamp of claim 1 wherein the elongate core is formed from a super elastic material.

9. A surgical clamp assembly for use in keyhole surgery, the assembly comprising:
a surgical clamp for forming a clamping loop around a bodily lumen, the clamp comprising an elongate body;
a deployment tube for deploying the clamp through a keyhole; and
a manipulator for manipulating the clamp through the deployment tube,
wherein the elongate body is biased to move from a constrained generally straight condition within the deployment tube to an unrestrained curled condition within a patient.

10. The assembly of claim 9 wherein the clamp comprises:
a leading portion comprising a gripper, the gripper having an open mouth gripably engageable with the elongate body to form the clamping loop; and
a trailing portion, the elongate body joining the trailing portion to the leading portion,
wherein the elongate body is biased to move from a constrained generally straight condition within a delivery device to an unrestrained curled condition within a patient.

11. The assembly of claim 10 wherein the open mouth of the gripper is configured to allow lateral entry of the elongate body into the gripper.

12. The assembly of claim 11 wherein the open mouth of the gripper is configured to provide a higher degree of slip resistance against movement of the elongate body in a direction loosening the clamping loop than the degree of slip resistance against movement of the elongate body in a direction tightening the clamping loop.

13. The assembly of claim 12 wherein the open mouth includes a tooth and a face opposing the tooth together defining an opening, wherein the opening is sized to grippingly receive the elongate body.

14. The assembly of claim 13 comprising a latching arrangement configured to latch the clamp to the manipulator.

15. The assembly of claim 14 wherein the latching arrangement comprises a first latch surface on the manipulator cooperable with a second latch surface on the clamp,
wherein the first and second latching surfaces remain engaged while the second latch surface remains inside the deployment tube and wherein the first and second latching surfaces readily disengage when the second latch surface is outside the deployment tube.

16. The assembly of claim 15 wherein the first latch surface is located on a latching projection that projects from the manipulator and the second latch surface is located within a latching slot within the clamp.

17. The assembly of claim 16 wherein the latching arrangement comprises a locking arrangement, the locking arrangement configured to prevent substantial axial movement of the manipulator in a direction axial to the deployment tube to prevent unintended release of the clamp.

18. The assembly of claim 17 wherein the tooth is asymmetrically shaped so as to provide the higher degree of slip resistance against movement of the elongate body in a direction loosening the clamping loop than the degree of slip resistance against movement of the elongate body in a direction tightening the clamping loop.

19. The assembly of claim 18 wherein the elongate body has an external shape that defines a series of ratchet teeth, the ratchet teeth being cooperable with the tooth of the mouth to form a ratchet.

20. The assembly of claim 12 wherein the elongate body comprises an elongate deformable layer having a first hardness and an elongate core having a second hardness, the first hardness less than the second hardness.
